(19) United States
(12) Patent Application Publication
(10) Pub. No.: US 2023/0147573 A1
(43) Pub. Date: May 11, 2023

(54) SENSOR DEVICE-BASED DETERMINATION OF GEOGRAPHIC ZONE DISPOSITIONS

(71) Applicant: AT&T Intellectual Property I, L.P., Atlanta, GA (US)

(72) Inventors: Ginger Chien, Bellevue, WA (US); Zhi Cui, Sugar Hill, GA (US); Eric Zavesky, Austin, TX (US); Robert T. Moton, Jr., Alpharetta, GA (US); Adrianne Binh Luu, Atlanta, GA (US); Robert Koch, Peachtree Corners, GA (US)

(21) Appl. No.: 17/524,521

(22) Filed: Nov. 11, 2021

(51) Int. Cl. G06Q 30/02; G06K 9/00; G06K 9/62; G10L 25/63; G06N 20/00

(52) U.S. Cl. CPC G06Q 30/0205; G06K 9/00228; G06K 9/00268; G06K 9/6256; G06K 9/6288; G10L 25/63; G06N 20/00; G06K 9/00302; G01N 33/18

(57) ABSTRACT

A processing system may collect sensor data for a first zone via sensor devices deployed in the first zone in communication with the processing system, the sensor devices including at least one of a camera or a microphone, and where the sensor data is collected over a period of time, may identify that a first disposition is associated with the first zone based upon the sensor data, by applying at least one detection model configured to output at least one disposition based upon the sensor data as input data, where the at least one disposition comprises the first disposition, where the sensor data comprises a plurality of inputs to the at least one detection model, and where the identifying comprises aggregating a plurality of outputs of the at least one detection model from the plurality of inputs, and may report that the first disposition is associated with the first zone.

The present disclosure relates generally to network-connected sensor devices, and more particularly to methods, computer-readable media, and apparatuses for reporting a disposition of a first zone identified based upon sensor data from a plurality of sensor devices applied to at least one detection model.

### BACKGROUND

Current trends in wireless technology are leading towards a future where virtually any object can be network enabled and Internet Protocol (IP) addressable. The pervasive presence of wireless networks, including cellular, Wi-Fi, ZigBee, satellite and Bluetooth networks, and the migration to a 128-bit IPv6-based address space provides the tools and resources for the paradigm of the Internet of Things (IoT) to become a reality. In addition, the household use of various sensor devices is increasingly prevalent. These sensor devices may relate to biometric data, environmental data, premises monitoring, and so on.

### SUMMARY

In one example, the present disclosure describes a method, computer-readable medium, and apparatus for reporting a disposition of a first zone identified based upon sensor data from a plurality of sensor devices applied to at least one detection model. For example, a processing system including at least one processor may collect sensor data for a first zone via a plurality of sensor devices deployed in the first zone in communication with the processing system, where the plurality of sensor devices comprises at least one of a camera or a microphone, and where the sensor data is collected over a period of time. The processing system may next identify that a first disposition is associated with the first zone based upon the sensor data, where the identifying comprises applying at least one detection model to the sensor data, where the at least one detection model is configured to output at least one disposition based upon the sensor data as input data to the at least one detection model, and where the at least one disposition comprises the first disposition. The sensor data collected over the period of time may comprise a plurality of inputs to the at least one detection model, and the identifying that the first disposition is associated with the first zone may include aggregating a plurality of outputs of the at least one detection model from the plurality of inputs. The processing system may then report that the first disposition is associated with the first zone.

### BRIEF DESCRIPTION OF THE DRAWINGS

The teachings of the present disclosure can be readily understood by considering the following detailed description in conjunction with the accompanying drawings, in which:

FIG. 1 illustrates an example network related to the present disclosure;

FIG. 2 illustrates examples of presenting zone disposition profiles, in accordance with the present disclosure;

FIG. 3 illustrates a flowchart of an example method for reporting a disposition of a first zone identified based upon sensor data from a plurality of sensor devices applied to at least one detection model; and

FIG. 4 illustrates a high level block diagram of a computing device specifically programmed to perform the steps, functions, blocks and/or operations described herein.

To facilitate understanding, identical reference numerals have been used, where possible, to designate identical elements that are common to the figures.

### DETAILED DESCRIPTION

Examples of the present disclosure provide for methods, computer-readable media, and apparatuses for reporting a disposition of a first zone identified based upon sensor data from a plurality of sensor devices applied to at least one detection model. For instance, examples of the present disclosure collect sensor data that is used to identify a specific geographic zone as having certain dispositions, e.g., character or personality features, such as representative moods or emotional states of its population. The determination of the disposition is not for specific people within the geographic zone, but rather for the geographic zone as a whole, e.g., based on the activities and behavior of people within the zone as determined from sensor data (e.g., image data from cameras and/or audio data from microphones), and in one example from other sensor data collected within or proximate to the zone.

By identifying contiguous or non-contiguous points (e.g., a geographic zone) with common disposition, or “personality” characteristics, the resulting disposition(s) that is/are determined may be used by city planners, real estate agents, advertisers, and others who may need to better understand characteristics of the zones within an area, the different needs of each zone, and so forth. The data collected may be used to serve as a proxy for describing certain personality traits of the population of a geographic zone. A zone may be defined by a collection of geographic coordinates that are contiguous. A zone may be one of several or one of many zones in an area (e.g., a neighborhood within a city, or the like).

There may be several types of network-connected sensor devices that are deployed within the zone or in the overall area that may record and/or detect various aspects of the environment. In one example, the present disclosure may utilize image data from video and/or still cameras. In one example, the present disclosure may alternatively or additionally use audio data from microphones deployed throughout the zone. In one example, sensor data from additional sensor devices, e.g., secondary or supplemental sensor data sources, may be used in determining one or more dispositions of a zone. For instance, these secondary sensor devices may include air quality sensors, water quality sensors, infrastructure vibration sensors (such as attached to buildings or bridges), olfactory sensors, and others. All of these sensor devices may be networked and may report collected data on request or periodically to be stored in a sensor database.

The sensor data collected may be associated with different dispositions. For instance, the level of vibration in a bridge, if high, may be interpreted as having a large amount of traffic, having a large amount of heavy vehicle traffic, e.g., delivery trucks and construction vehicles, having structural issues such as maintenance issues, aging issues, and so on. In turn, this sensor data may indicate a “stressed” disposition, e.g., there may be persistent traffic congestion issues, major construction events nearby, poorly maintained infrastructures, and the like.

Likewise, water quality readings may be used as a proxy for sensitivity or pride. For instance, a city (or a neighborhood or other zones therein) may have a higher level of pride if water quality or air quality readings are favorable.

Likewise, motion sensors may serve as a proxy for a level of extroversion or friendliness within a zone, e.g., when also combined with microphone readings from a nearby location. For instance, if a high level of motion is detected in a zone and is accompanied by laughter or play as determined based on an analysis of microphone readings or video camera recordings, the conclusion may be that the level of “extroversion” or “friendliness” is high in the area. Microphone, video camera, and motion detector sensor data may also indicate public safety levels which may be interpreted as a proxy for the trait of “contentment.” For instance, detection of screams or loud arguments with use of inappropriate or foul language, gun fire, or other distress sounds (e.g., police sirens, ambulance sirens, or fire truck sirens) may be used to interpret that the zone is low in terms of public safety, and therefore also low in “contentment” or “pride.”

Audio data may also be collected anonymously and analyzed to determine dialogue used or terms used that may be indicative of a disposition of the zone. For instance, audio data may be used to determine that various people in a zone are tourists (e.g., detection of a spoken foreign language, detection of a discussion of a known tourist site, etc.), or are young people based on dialogue used or the frequency of their voices. Dialogue analysis may also be used to estimate the temperament or level of happiness of people in an area. For instance, if statements that may be detected as complaints prevail, the level of happiness may be low. Similarly, video analysis may be used to estimate a level of pace in an area. For instance, if people are walking or running at a fast pace, the video analysis may attempt to distinguish between people who are walking at a fast pace to work, which may be a representation of a busy, fast-paced environment (e.g., people with business attire, people carrying briefcases or backpacks, people moving large packages, people pushing a hand truck, etc.), in contrast to people who are detected to be running at a fast pace (e.g., joggers in T-shirts and shorts, joggers with running shoes, etc.), which may be indicative of an active, vibrant, exercise-conscious community. It should be noted that in one example the present disclosure utilizes the sensor data for the sole purpose of identifying dispositions of zones and does not store audio or video data for any longer than necessary for such purpose. In addition, the image or audio data is not used to personally identify any specific individuals or to create a record of any words or actions.

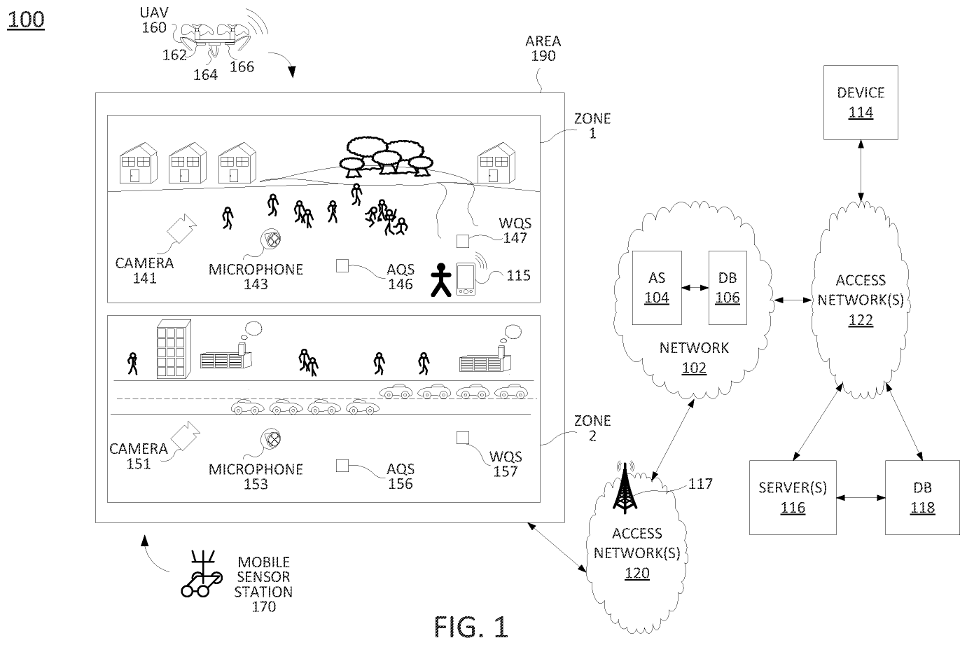

Results of determined disposition(s) may be aggregated and presented on a map or in other formats for consumption by a user or analyst, e.g., indicating for one or more areas, one or more dispositions that are determined, indicating zone having a particular disposition (or not) (e.g., zones having a common, or shared characteristic/trait), and so forth. This knowledge may be associated with other information, such as information on the estimated numbers of people in a zone, density of people in a zone, indications of whether people in a zone are regularly present in a zone or are considered to be temporary visitors in a zone, and so forth. For instance, if there is a perceived low contentment in a zone that is typically associated with tourists, this may be informative to city planners or a tourist bureau. Likewise, other dispositions may be mapped. For instance, a high friendliness score may indicate a high friendliness zone, which may also be informative. These and other aspects of the present disclosure are discussed in greater detail below in connection with the examples of FIGS. 1-4.

To further aid in understanding the present disclosure, FIG. 1 illustrates an example system 100 in which examples of the present disclosure for reporting a disposition of a first zone identified based upon sensor data from a plurality of sensor devices applied to at least one detection model may operate. The system 100 may include any one or more types of communication networks, such as a traditional circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wireless network, a cellular network (e.g., 2G, 3G, 4G, 5G and the like), a long term evolution (LTE) network, and the like, related to the current disclosure. It should be noted that an IP network is broadly defined as a network that uses Internet Protocol to exchange data packets. Additional example IP networks include Voice over IP (VoIP) networks, Service over IP (SoIP) networks, and the like.

In one example, the system 100 may comprise a network 102, e.g., a core network of a telecommunication network. The network 102 may be in communication with one or more access networks 120 and 122, and the Internet (not shown). In one example, network 102 may combine core network components of a cellular network with components of a triple play service network; where triple-play services include telephone services, Internet services and television services to subscribers. For example, network 102 may functionally comprise a fixed mobile convergence (FMC) network, e.g., an IP Multimedia Subsystem (IMS) network. In addition, network 102 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. Network 102 may further comprise a broadcast television network, e.g., a traditional cable provider network or an Internet Protocol Television (IPTV) network, as well as an Internet Service Provider (ISP) network. In one example, network 102 may include a plurality of television (TV) servers (e.g., a broadcast server, a cable head-end), a plurality of content servers, an advertising server (AS), an interactive TV/video-on-demand (VoD) server, and so forth. For ease of illustration, various additional elements of network 102 are omitted from FIG. 1.

In one example, the access networks 120 and 122 may comprise Digital Subscriber Line (DSL) networks, public switched telephone network (PSTN) access networks, broadband cable access networks, Local Area Networks (LANs), wireless access networks (e.g., an IEEE 802.11/Wi-Fi network and the like), cellular access networks, 3^{rd} party networks, and the like. For example, the operator of network 102 may provide a cable television service, an IPTV service, or any other types of telecommunication service to subscribers via access networks 120 and 122. In one example, the access networks 120 and 122 may comprise different types of access networks, may comprise the same type of access network, or some access networks may be the same type of access network and other may be different types of access networks. In one example, the network 102 may be operated by a telecommunication network service provider. The network 102 and the access networks 120 and 122 may be operated by different service providers, the same service provider or a combination thereof, or may be operated by entities having core businesses that are not related to telecommunications services, e.g., corporate, governmental or educational institution LANs, and the like. In one example, each of access networks 120 and 122 may include at least one access point, such as a cellular base station, non-cellular wireless access point, a digital subscriber line access multiplexer (DSLAM), a cross-connect box, a serving area interface (SAI), a video-ready access device (VRAD), or the like, for communication with various endpoint devices. For instance, as illustrated in FIG. 1, access network(s) 120 include a wireless access point 117 (e.g., a cellular base station).

In one example, the access networks 120 may be in communication with various devices or computing systems/processing systems, such as mobile device 115, camera 141, camera 151, microphone 143, microphone 153, air quality sensor (AQS) 146, AQS 156, water quality sensor (WQS) 147, WQS 157, uncrewed aerial vehicle (UAV) 160, mobile sensor station 170, and so forth. Similarly, access networks 122 may be in communication with one or more devices, e.g., device 114, server(s) 116, database(s) (DB(s)) 118, etc. Access networks 120 and 122 may transmit and receive communications between mobile device 115, camera 141, camera 151, microphone 143, microphone 152, air quality sensor (AQS) 146, AQS 156, water quality sensor (WQS) 147, WQS 157, UAV 160, mobile sensor station 170, device 114, and so forth, and server(s) 116 and/or DB(s) 118, application server (AS) 104 and/or database (DB) 106, other components of network 102, devices reachable via the Internet in general, and so forth.

In one example, device 114 may comprise a mobile device, a cellular smart phone, a laptop, a tablet computer, a desktop computer, a wearable computing device (e.g., a smart watch, a smart pair of eyeglasses, etc.), an application server, a bank or cluster of such devices, or the like. Similarly, mobile device 115 may comprise a cellular smart phone, a laptop, a tablet computer, a wearable computing device (e.g., a smart watch, a smart pair of eyeglasses, etc.), or the like. In accordance with the present disclosure, mobile device 115 may include one or more sensors for tracking location, speed, distance, altitude, or the like (e.g., a Global Positioning System (GPS) unit), for tracking orientation (e.g., gyroscope and compass), and so forth. Cameras 141 and 151 may comprise publicly deployed cameras such as traffic cameras, security cameras, and so forth. Microphones 143 and 153, air quality sensors 146 and 156, and water quality sensors 147 and 157 may similarly be network-connected “Internet of Things” (IoT) devices. Although omitted from FIG. 1, for ease of illustrate, sensor devices may also include humidity sensors, thermometers, rain sensors, motion detectors, vibration detectors, and so forth.

In accordance with the present disclosure, sensor devices may include mobile sensors. For instance, FIG. 1 illustrates an uncrewed aerial vehicle (UAV) 160 and mobile sensor station 170. In accordance with the present disclosure, UAV 160 may include a camera 162 and one or more radio frequency (RF) transceivers 166 for cellular communications and/or for non-cellular wireless communications. In one example, UAV 160 may also include one or more module(s) 164 with one or more sensors or additional controllable components, such as one or more infrared, ultraviolet, and/or visible spectrum light sources, a light detection and ranging (LiDAR) unit, a radar unit, a microphone, a speaker, and so forth. Mobile sensor station 170 may be similarly equipped with one or more radio frequency (RF) transceivers for cellular communications and/or for non-cellular wireless communications, and one or more sensors, such as one or more cameras or other light sensors, one or more microphones, an air quality sensor, a water quality sensor, a humidity sensor, a thermometer, a rain sensor, and so forth.

In one example, each of these sensor devices (camera 141, camera 151, microphone 143, microphone 153, air quality sensor (AQS) 146, AQS 156, water quality sensor (WQS) 147, WQS 157, UAV 160, mobile sensor station 170) may communicate independently with access networks 120. In another example, one or more of these sensor devices may comprise a peripheral device that may communicate with remote devices, servers, or the like via access networks 120, network 102, etc. via another endpoint device, such as a gateway or router, or the like. Thus, one or more of the camera 141, camera 151, microphone 143, microphone 153, etc. may have a wired or wireless connection to another local device that may have a connection to access networks 120.

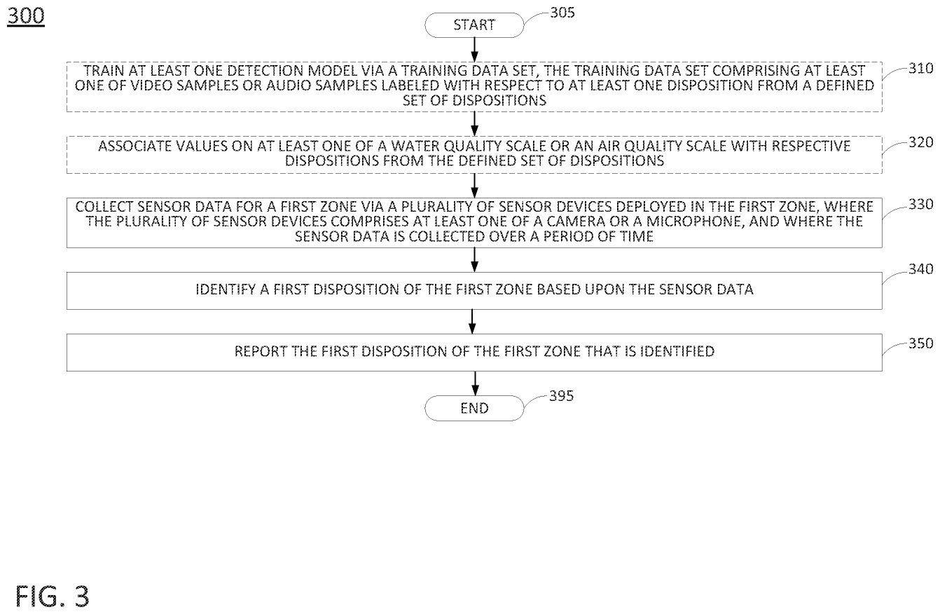

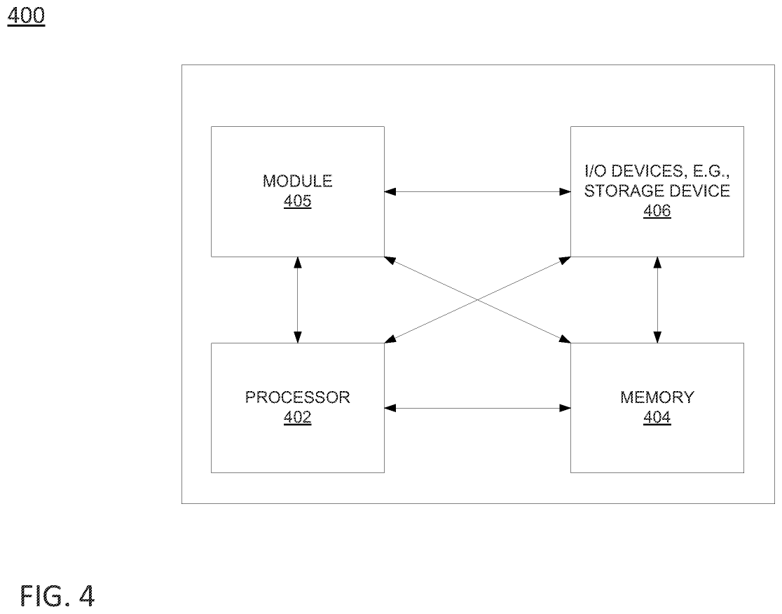

In one example, device 114 may include an application (app) for geographic disposition information, and which may establish communication with server(s) 116 to access disposition information regarding zones or areas, and so forth. For instance, as illustrated in FIG. 1, access networks 122 may be in communication with one or more servers 116 and one or more databases (DB(s)) 118. Alternatively, or in addition, device 114 may have a web browser via which a geographic disposition information website may be accessed, and via which disposition information regarding various zones or areas may be obtained. In accordance with the present disclosure, each of the server(s) 116 may comprise a computing system or server, such as computing system 400 depicted in FIG. 4, and may individually or collectively be configured to perform operations or functions for reporting a disposition of a first zone identified based upon sensor data from a plurality of sensor devices applied to at least one detection model (such as illustrated and described in connection with the example method 300 of FIG. 3). For instance, server(s) 116 may host a geographic disposition information website via which disposition data may be requested and presented to requesting devices.

It should be noted that as used herein, the terms “configure,” and “reconfigure” may refer to programming or loading a processing system with computer-readable/computer-executable instructions, code, and/or programs, e.g., in a distributed or non-distributed memory, which when executed by a processor, or processors, of the processing system within a same device or within distributed devices, may cause the processing system to perform various functions. Such terms may also encompass providing variables, data values, tables, objects, or other data structures or the like which may cause a processing system executing computer-readable instructions, code, and/or programs to function differently depending upon the values of the variables or other data structures that are provided. As referred to herein a “processing system” may comprise a computing device including one or more processors, or cores (e.g., as illustrated in FIG. 4 and discussed below) or multiple computing devices collectively configured to perform various steps, functions, and/or operations in accordance with the present disclosure.

In one example, DB(s) 118 may comprise one or more physical storage devices integrated with server(s) 116 (e.g., a database server), attached or coupled to the server(s) 116, or remotely accessible to server(s) 116 to store various types of information in support of systems for reporting a disposition of a first zone identified based upon sensor data from a plurality of sensor devices applied to at least one detection model, in accordance with the present disclosure. For example, DB(s) 118 may include a sensor database to store a record for each sensor that may include: a sensor identifier (ID), a network address of the sensor, sensor owner information, a sensor type and/or the type(s) of data the sensor is capable of collecting, a fixed location (for a non-mobile sensor), the sensor availability (e.g., dates, data or time ranges, etc.), and for a mobile sensor, the sensor's range, operating time (e.g., without recharging or refueling, etc.), a current location, and so on. DB(s) 118 may also temporally store collected sensor data (e.g., in the sensor database or in a separate database). In addition, DB(s) 118 may comprise one or more geographic databases, e.g., storing maps and/or geographic data sets. For instance, DB(s) 118 may store a map/geographic data set for area 190, which may include information regarding zones 1 and 2, such as boundary point/coordinate sets or similar descriptors. In one example, DB(s) 118 may also store a database of detection models, e.g., machine learning models (MLMs) or the like, for detecting semantic content in video and/or audio data, for associating sensor data to dispositions, and so forth.

In an illustrative example, server(s) 116 may generate a disposition profile of zone 1 in accordance with one or more dispositions of zone 1 determined based upon sensor data from various sensor devices, e.g., including at least one of camera 141 or microphone 143, and in one example further including AQS 146 and/or WQS 147, sensor data from sensors of UAV 160 and/or mobile sensor station 170, and so forth. For instance, camera 141 may collect image data (e.g., video and/or still images) which may appear to include a number of people gathered in a park playing baseball. In one example, server(s) 116 may apply detection models (e.g., MLMs or the like stored in DB(s) 118) for detecting semantic content in video, such as “baseball,” “exercise,” “crowd,” “car,” “traffic,” etc. In one example, the semantic content may be mapped to various dispositions from a defined set of dispositions. For instance, detected “sports” and “recreation” can be mapped to one or more dispositions of “vibrant,” “healthy,” etc. In another example, server(s) 116 may apply detection models for semantic content, where the semantic content comprises the dispositions from the defined set of dispositions. For instance, server(s) 116 may deploy detection models for dispositions of: “sensitive,” “proud,” “extroverted,” “friendly,” “curious,” “dour,” “content,” “restless,” “driven,” “relaxed,” “uptight,” and so forth. For example, in such case, the dispositions may be identified more directly from the captured image data using detection models, without intermediate determination of other types of semantic content and then mapping into associated dispositions. In one example, server(s) 116 may apply detection models for detecting human faces (and emotional states thereof) in image data from camera 141 or the like. In one example, the emotional states may comprise dispositions from the defined set of dispositions. In another example, the emotional states may comprise a larger set of emotional states that may be mapped to dispositions from the defined set of dispositions.

In one example, dispositions detected as described above may relate to one or more disposition/mood scales and may be aggregated with other detected dispositions. For instance, a disposition scale for “happiness” may comprise “happy” on one end and “sad” on the other end, with “neutral” in the middle and possible additional levels of “very happy,” “extremely happy,” “very sad,” “extremely sad,” or the like. In one example, a disposition representative of a zone may be moved up or down the scale depending on the detected disposition in a particular instance of sensor data. For instance, a detected disposition of “happy” in an instance of image data from camera 141 may move the overall disposition for zone 1 relating to the happiness scale toward the happiness end. However, a subsequent detected disposition of “sad” in an instance of image data from camera 141 may move the overall disposition of zone 1 back to neutral on a “happiness” disposition scale.

In one example, a detected disposition may be weighted differently depending upon the manner in which the disposition is detected. For instance, a detected disposition of “happy” in a single face may be weighted less than a detected disposition of “happy” in semantic content comprising a “party.” In one example, disposition of zones may be quantified along multiple disposition scales. For instance, disposition scales may relate to Profile of Mood States (POMS) six mood subscales (tension, depression, anger, vigor, fatigue, and confusion) or a similar set of Positive Activation-Negative Activation (PANA) model subscales. It should be noted that in the PANA model, there are negative subscales and positive subscales. Thus, an instance of a detection of a disposition relating to a particular subscale in sensor data for a zone may cause a tally for that subscale to be increased (e.g., rather than moving up or down a POMS mood subscale, for instance). In one example, a disposition of a zone may be a metric or score relating to a tally or count of a number or percentage of instances in which sensor data from the zone is indicative of the disposition. In one example, dispositions for which a tally, count, or percentage of instances on an associated subscale exceed a threshold may be reported as dispositions of the zone in a zone disposition profile (e.g., characteristic dispositions). It should be noted that the foregoing are just two examples of mood/emotional state models and associated scales (e.g., providing a defined set of possible dispositions), and that other scales may be devised in accordance with the present disclosure.

With respect to determining a disposition from facial images, server(s) 116 may quantify the extent to which an image matches various dispositions. For instance, a current image may be quantized and evaluated to determine how closely the current image matches to eigenfaces (or other detection models) of various dispositions, or moods (e.g., the respective distances in the feature space). In other words, server(s) 116 may not determine a single mood that best characterizes a facial image, but may obtain a value for each mood that indicates how well the image matches to a mood. In one example, the distance determined for each mood may be matched to a mood scale (e.g., “not at all,” “a little bit,” “moderately,” “quite a lot,” such as according to the POMS methodology). In addition, each level on the mood scale may be associated with a respective value (e.g., ranging from zero (0) for “not at all” to (4) for “quite a lot”). In one example, server(s) 116 may determine an overall level to which a zone exhibits a particular disposition (and for multiple possible dispositions) in accordance with the values determined for dispositions (and/or for various moods, mental states, and/or emotional states). For example, server(s) 116 may sum values for negative moods/subscales and subtract this total from a sum of values for positive moods/subscales from multiple instances of image data from camera 141 or the like. Alternatively, or in addition, server(s) 116 may calculate scores for certain subscales (e.g., tension, depression, anger, fatigue, confusion, vigor, or the like) comprising composites of different values for component mental states, moods, or emotional states.

In the case of image or audio data, in one example DB(s) 118 may store and server(s) 116 may apply various semantic content detection models, e.g., MLMs or other detection models, for identifying relevant semantic content/features (e.g., dispositions or other semantic content) within the image and/or audio data. For example, in order to detect semantic content of “baseball game” in image data, server(s) 116 may deploy a detection model (e.g., stored in DB(s) 118). This may include one or more images of baseball games (e.g., from different angles, in different scenarios, etc.), and may alternatively or additionally include feature set(s) derived from one or more images and/or videos of baseball games, respectively. For instance, DB(s) 118 may store a respective scale-invariant feature transform (SIFT) model, or a similar reduced feature set derived from image(s) of baseball games, which may be used for detecting additional instances of baseball games in image data via feature matching. Thus, in one example, a feature matching detection algorithm/model stored in DB(s) 118 may be based upon SIFT features. However, in other examples, different feature matching detection models/algorithms may be used, such as a Speeded Up Robust Features (SURF)-based algorithm, a cosine-matrix distance-based detector, a Laplacian-based detector, a Hessian matrix-based detector, a fast Hessian detector, etc.

The visual features used for detection of “baseball game” or other semantic content (such as different types of dispositions, objects/items, events, weather, actions, occurrences, etc.) may include low-level invariant image data, such as colors (e.g., RGB (red-green-blue) or CYM (cyan-yellow-magenta) raw data (luminance values) from a CCD/photo-sensor array), shapes, color moments, color histograms, edge distribution histograms, etc. Visual features may also relate to movement in a video and may include changes within images and between images in a sequence (e.g., video frames or a sequence of still image shots), such as color histogram differences or a change in color distribution, edge change ratios, standard deviation of pixel intensities, contrast, average brightness, and the like.

In one example, server(s) 116 may perform an image salience detection process, e.g., applying an image salience model and then performing an image recognition algorithm over the “salient” portion of the image(s) or other image data/visual information, such as from camera 141 or the like. Thus, in one example, visual features may also include a length to width ratio of an object, a velocity of an object estimated from a sequence of images (e.g., video frames), and so forth. Similarly, in one example, server(s) 116 may apply an object/item detection and/or edge detection algorithm to identify possible unique items in image data (e.g., without particular knowledge of the type of item; for instance, the object/edge detection may identify an object in the shape of a person in a video frame, without understanding that the object/item is a person). In this case, visual features may also include the object/item shape, dimensions, and so forth. In such an example, object/item recognition may then proceed as described above (e.g., with respect to the “salient” portions of the image(s) and/or video(s)).

It should be noted that as referred to herein, a machine learning model (MLM) (or machine learning-based model) may comprise a machine learning algorithm (MLA) that has been “trained” or configured in accordance with input training data to perform a particular service, e.g., to detect a perceived disposition, a perceived mental state, mood, or emotional state, or other semantic content, or a value indicative of such a perceived disposition, mental state, mood, etc. In one example, MLM-based detection models associated with image data inputs may be trained using samples of video or still images that may be labeled by participants or by human observers with dispositions (and/or with other semantic content labels/tags). For instance, a machine learning algorithm (MLA), or machine learning model (MLM) trained via a MLA may be for detecting a single semantic concept, such as a disposition, or may be for detecting a single semantic concept from a plurality of possible semantic concepts that may be detected via the MLA/MLM (e.g., a set of dispositions). For instance, the MLA (or the trained MLM) may comprise a deep learning neural network, or deep neural network (DNN), such as convolutional neural network (CNN), a generative adversarial network (GAN), a support vector machine (SVM), e.g., a binary, non-binary, or multi-class classifier, a linear or non-linear classifier, and so forth. In one example, the MLA may incorporate an exponential smoothing algorithm (such as double exponential smoothing, triple exponential smoothing, e.g., Holt-Winters smoothing, and so forth), reinforcement learning (e.g., using positive and negative examples after deployment as a MLM), and so forth. It should be noted that various other types of MLAs and/or MLMs, or other detection models may be implemented in examples of the present disclosure such as a gradient boosted decision tree (GBDT), k-means clustering and/or k-nearest neighbor (KNN) predictive models, support vector machine (SVM)-based classifiers, e.g., a binary classifier and/or a linear binary classifier, a multi-class classifier, a kernel-based SVM, etc., a distance-based classifier, e.g., a Euclidean distance-based classifier, or the like, a SIFT or SURF features-based detection model, as mentioned above, and so on. In one example, MLM-based detection models may be trained at a network-based processing system (e.g., server(s) 116) and deployed to sensor devices, such as cameras 141 and 151, microphones 143 and 153, etc.). Similarly, non-MLM-based detection models may be generated by server(s) 116, e.g., based upon feature sets from sample input data as described above. It should also be noted that various pre-processing or post-recognition/detection operations may also be applied. For example, server(s) 116 may apply an image salience algorithm, an edge detection algorithm, or the like (e.g., as described above) where the results of these algorithms may include additional, or pre-processed input data for the one or more detection models.

Similarly, server(s) 116 may generate, store (e.g., in DB(s) 118), and/or use various speech or other audio detection models, which may be trained from extracted audio features from one or more representative audio samples, such as low-level audio features, including: spectral centroid, spectral roll-off, signal energy, mel-frequency cepstrum coefficients (MFCCs), linear predictor coefficients (LPC), line spectral frequency (LSF) coefficients, loudness coefficients, sharpness of loudness coefficients, spread of loudness coefficients, octave band signal intensities, and so forth, wherein the output of the model in response to a given input set of audio features is a prediction of whether a particular semantic content is or is not present (e.g., sounds indicative of a particular disposition (e.g., “excited,” “stressed,” “content,” “indifferent,” etc.), the sound of breaking glass (or not), the sound of rain (or not), etc.). For instance, in one example, each audio model may comprise a feature vector representative of a particular sound, or a sequence of sounds.

It is also noted that detection models may be associated with detecting dispositions or other moods, mental states, and/or emotional states from facial images. For instance, such detection models may include eignefaces representing various dispositions or other moods, mental states, and/or emotional states, or similar SIFT or SURF models. For instance, a quantized vector, or set of quantized vectors representing a disposition or other moods, mental states, and/or emotional states in facial images may be encoded using techniques such as principal component analysis (PCA), partial least squares (PLS), sparse coding, vector quantization (VQ), deep neural network encoding, and so forth. Thus, in one example, server(s) 116 may employ a feature matching detection algorithm such as described above. For instance, in one example, server(s) 116 may obtain new content and may calculate the Euclidean distance, Mahalanobis distance measure, or the like between a quantized vector of the facial image data in the content and the feature vector(s) of the detection model(s) to determine if there is a best match (e.g., the shortest distance) or a match over a threshold value.

It is again noted that dispositions may include a defined set of positive dispositions (e.g., moods/mental states/emotional states such as, happy, excited, relaxed, content, calm, cheerful, optimistic, pleased, blissful, amused, refreshed, or satisfied), negative dispositions (such as, sad, angry, upset, devastated, mad, hurt, sulking, depressed, annoyed, or enraged), and neutral dispositions (such as indifferent, bored, sleepy, and so on). In addition, detection models for semantic content may include other types of semantic content that are not necessarily dispositions, or moods/emotional states/mental states, such as “sports,” “recreation,” “concert,” “traffic,” “argument,” “fight,” etc., which can then be mapped to respective dispositions. For instance, the mapping may include a word association graph, sematic map, or the like, where connections and edge weights may be used to sum and quantify the extent to which an instance of image data may be indicative of one or more dispositions (e.g., semantic concepts in image data may be detected as “sports” and “recreation,” where these terms may be linked to one or more terms representative of one or more dispositions (e.g., “vibrant,” “happy,” etc.) in a word association graph and/or semantic map).

In the example of FIG. 1, server(s) 116 may therefore obtain image data from camera 141. Server(s) 116 may then identify semantic concepts such as “baseball,” “exercise,” etc. which may be mapped to one or more dispositions, and/or may identify one or more disposition(s) in the image data via detection models for such disposition(s). For instance, this may be applied holistically with respect to the image data, or may be based upon dispositions or other moods, mental states, and/or emotional states identified in facial images present within the image data from camera 141. Similarly, server(s) may identify one or more dispositions, or other semantic content that may be mapped to one or more dispositions, in audio data from microphone 143 (e.g., the sounds of “laughter,” the sounds of “argument,” the sounds of being “stressed” such as grunting or groaning noise, the sounds of being “excited,” etc.). In addition, server(s) 116 may aggregate these detected disposition(s) to determine a disposition profile of zone 1, such as to identify that zone 1 is predominantly “vibrant,” “active,” “relaxed,” “content,” or the like. These dispositions are only illustrative and should not be interpreted to be an exhaustive list.

It should be noted that server(s) 116 may also utilize image data and/or audio data from other sensor devices, such as additional cameras, additional microphones, camera 162 or other sensors of UAV 160 and/or mobile sensor station 170, and so forth to identify and aggregate additional dispositions. Similarly, server(s) 116 may also generate a disposition profile of zone 2 using similar image data and/or audio data from camera 151, microphone 153, and/or other cameras or microphones, camera 162 or other sensors of UAV 160 and/or mobile sensor station 170, and so forth to identify and aggregate dispositions with regard to zone 2. For instance, server(s) 116 may determine that zone 2 is predominantly “stressed,” “angry,” “despondent,” “fearful,” or the like. For example, the image data from camera 151 may contain semantic concepts of “argument,” such as two of the people present in zone 2 having an argument. Similarly, audio data from microphone 153 may capture the sounds of an argument, may capture the sound of car horns honking in a traffic jam, and so forth. In one example, the image data from camera 151 may include people walking fast, but server(s) 116 may determine via the outputs of one or more semantic concept detection models that the image data does not reflect “exercise” or “jogging,” but rather shows a semantic concept of people “rushing to work” (which may then be mapped to one or more dispositions, such as “stressed”).

As discussed above, in one example, the present disclosure may utilize additional sensor data to help identify zone dispositions. For example, the present disclosure may learn and correlate dispositions from areas and/or zones in which image and audio data are widely available to other types of sensor data as predictors. Then in new areas where there may be less available audio or image data, the other types of sensor data may be more heavily relied upon as predictors. In one example, the present disclosure may learn relationships between dispositions and values of these other types of sensor data via resident surveys and/or person-on-the-street surveys or interviews (e.g., to capture profiles of visitors). In one example, individuals' dispositions may be used as proxies for dispositions of a zone, and these individuals may be used as training examples from which inferred dispositions may be learned from the predictors. In one example, the present disclosure may then examine other zones and determine disposition(s) using such other sensor data. Alternatively, or in addition, such additional sensor data may be used as secondary factors in conjunction with image and/or audio data for a zone.

For instance, in the example of FIG. 1, server(s) 116 may additionally obtain air quality data and water quality data from air quality sensor 146 and water quality sensor 147, respectively (and/or from other sensor devices (not shown), such as olfactory sensors, etc.). From such sensor data, server(s) 116 may then identify associated dispositions based upon the associative models described above. For instance, good water quality may be associated with dispositions of “content,” “active,” etc. Poor water quality may be associated with dispositions of “stressed,” “angry,” etc. In one example, server(s) 116 may determine a disposition profile for zone 1 based upon a weighting of dispositions determined from image and/or audio data on the one hand, and dispositions determined from supplemental sensor data on the other. For instance, the disposition(s) identified from such additional sensor data may be weighted less than disposition(s) identified from image data of camera 141 and/or audio data of microphone 143 in composing tallies or tracking a value along a disposition/mood scale. In one example, the weighting may be scaled based upon a quantity of image and/or audio data available. For instance, if no image or audio data is available, dispositions determined from additional sensor data may be weighted at 100 percent. If little image or audio data is available, dispositions determined from additional sensor data may be weighted at 60 percent. If a significant amount of image or audio data is available, dispositions determined from additional sensor data may be weighted at 20 percent, and so forth. Server(s) 116 may utilize additional sensor data from AQS 156 and WQS 157 similarly in determining a disposition profile for zone 2.

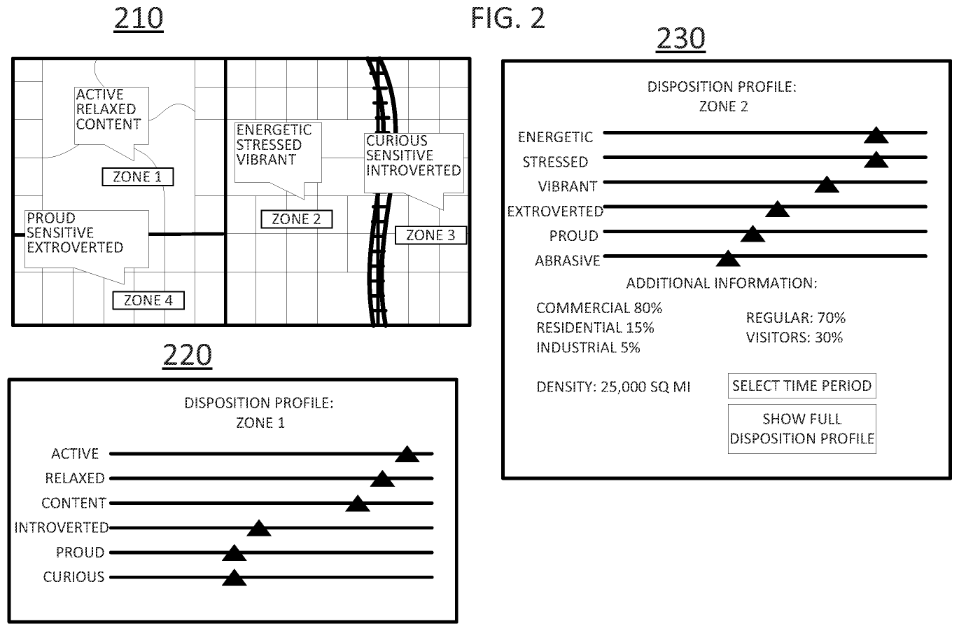

In one example, server(s) 116 may report zone disposition profiles to requesters. For example, a city planner may request a zone disposition profile of zone 1 from server(s) 116 via device 114. In one example, server(s) 116 may provide the zone disposition profile in one or several formats, such as via a map, in text form, in a chart form, and so forth. Examples of presenting zone disposition profiles are illustrated in FIG. 2 and discussed in greater detail below. In one example, server(s) 116 may report additional data regarding a zone, e.g., zone 1, along with a disposition profile, such as a type of area (e.g., residential, commercial, office, industrial, recreational, etc.). This can be taken explicitly from existing digital map data (e.g., stored in DB(s) 118 or otherwise obtained by server(s) 116), that may contain such labels, or can be learned from satellite images (e.g., using machine learning-based or other computer-implemented image classification techniques). The additional data may also include an estimated density of people in zone 1. This can be based upon existing available census and demographic data. Alternatively, or in addition, the density can be estimated in other ways, such as based on an average number of unique detected mobile endpoint devices in zone 1 (e.g., based upon connections to cellular base stations and/or Wi-Fi access points). For instance, the raw count of mobile devices may be mapped to an estimate of a total number of persons based upon past learning based methodologies. In one example, density of people may be broken down by morning, afternoon, evening, or hours of day, days of the week, days, months, seasons or other times of the year, and so forth.

In one example, the results may further include information on percentages of persons regularly present in zone 1 versus infrequent visitors. For instance, a detection of a same device (such as mobile device 115) over two or more days across at least two weeks may be considered to be a regular visitor, while a detection of a device for one or more days in a single week may be considered to be transient, unless also being detected in another week, or the like. This could misinterpret some visitors, such as an individual who regularly works in the zone and comes for one week at a time, once per month. However, the foregoing is merely illustrative of one way in which a delineation between regularly present and transient persons may be made. Thus, various other formulas may be used depending upon the data available with respect to endpoint devices. In one example, results may include information regarding multiple zones for which disposition information is requested, and/or for adjacent or nearby zones with respect to a zone for which disposition information is requested. For instance, server(s) 116 may provide a map of area 190 to device 114 with disposition profiles of both zone 1 and zone 2, or even a heat map with color coding.

It should be noted that the foregoing are just several examples of reporting a disposition of a first zone identified based upon sensor data from a plurality of sensor devices applied to at least one detection model, and that other, further, and different examples may be established in connection with the example of FIG. 1. It should also be noted that any number of server(s) 116 or database(s) 118 may be deployed. In one example, network 102 may also include an application server (AS) 104 and a database (DB) 106. In one example, AS 104 may perform the same or similar functions as server(s) 116. Similarly, DB 106 may store the same or similar information as DB(s) 118 (e.g., a sensor database, a zone disposition profile database, a database of semantic concept detection models, etc.). For instance, network 102 may provide a service to subscribing users and/or devices in connection with a geographic disposition information, e.g., in addition to television, phone, and/or other telecommunication services. In one example, AS 104, DB 106, server(s) 116, and/or DB(s) 118, or any one or more of such devices in conjunction with one or more of: mobile device 115, camera 141, camera 151, microphone 143, microphone 153, air quality sensor (AQS) 146, AQS 156, water quality sensor (WQS) 147, WQS 157, UAV 160, mobile sensor station 170, device 114, and so forth, may operate in a distributed and/or coordinated manner to perform various steps, functions, and/or operations described herein.

In addition, it should be noted that the system 100 has been simplified. Thus, the system 100 may be implemented in a different form than that which is illustrated in FIG. 1, or may be expanded by including additional endpoint devices, access networks, network elements, application servers, etc. without altering the scope of the present disclosure. In addition, system 100 may be altered to omit various elements, substitute elements for devices that perform the same or similar functions, combine elements that are illustrated as separate devices, and/or implement network elements as functions that are spread across several devices that operate collectively as the respective network elements. For example, the system 100 may include other network elements (not shown) such as border elements, routers, switches, policy servers, security devices, gateways, a content distribution network (CDN) and the like. Similarly, although only two access networks 120 and 122 are shown, in other examples, access networks 120 and/or 122 may each comprise a plurality of different access networks that may interface with network 102 independently or in a chained manner. For example, device 114 and server(s) 116 may be in communication with network 102 via different access networks, cameras 141 and 151 may be in communication with network 102 via different access networks, mobile sensor station 170 and UAV 160 may be in communication with network 102 via different access networks and so forth. Thus, these and other modifications are all contemplated within the scope of the present disclosure.

To further aid in understanding the present disclosure, FIG. 2 illustrates example screens of a user interface of a geographic disposition information service, in accordance with the present disclosure. For instance, example screens 210, 220, and 230 may be presented via a website or app for such a geographic disposition information service. In a first example screen 210, a map may be presented showing zones within an area, along with zone disposition information (e.g., disposition profiles) of the respective zones. For instance, the top of the dispositions for each of zones 1-4 may be presented as illustrated in the example screen 210. For example, the top dispositions may be those that are most representative of a zone based upon dispositions determined and aggregated over various instances of collected sensor data associated with the respective zones 1-4.

In one example, a requester, such a city planner, a prospective visitor to an area, a potential home purchaser, a prospective business owner, etc. may obtain disposition information on one or more zones in a different form and/or a more detailed form. For instance, the requester may click on a zone within the first example screen 210, such as zone 1, which may cause a more detailed disposition profile of zone 1 to be presented, such as illustrated in the second example screen 220. For instance, the second example screen 220 shows additional disposition information that may be representative of zone 1 (e.g., the top six dispositions). In addition, as can be seen in the second example screen, a relative level, score, or value of zone 1 as it relates to each of the dispositions is indicated along various scales. For instances, these levels/scores/values may be determined in a manner such as described above in connection with the example of FIG. 1, or the like.

Example screen 230 illustrates a further example of presenting a zone disposition profile using zone 2 as an example. For instance, the example screen 230 illustrates the top six dispositions for zone 2 (with relative levels/scores/values for each such disposition indicated). In addition, example screen 230 presents additional information regarding zone 2, such as demographic information (e.g., a profile of land-use types, a population density, information regarding percentages or those regularly present in zone 2 versus those who may be considered visitors/temporary, and so forth).

It should be noted that FIG. 2 illustrates just several examples of how area and zone disposition information may be presented in accordance with a geographic disposition information service of the present disclosure and that other, further, and different example screens and/or user interface(s) may be utilized in various designs. For instance, as illustrated in the example screen 230, a button may be included for “select time period,” which may allow a requester to obtain a disposition profile with regard to times of the day, days of the week, months, seasons of the year, and so forth. For example, a processing system of the present disclosure may generate different disposition profiles for a zone, such as zone 2, for such different time periods based upon historical sensor data collected during such respective time periods. As further illustrated in the example screen 230, a button may be included for “show full disposition profile,” which may allow a requester to view information regarding additional dispositions from a set of possible dispositions (e.g., dispositions beyond the top six that are shown in the example screen 230). In another example, a requester may select a type of disposition, and may be presented with a heat map of zones in an area that may exhibit the disposition. For instance, a requester may wish to see all zones in an area that are considered “stressed” and may be presented with a map that shades, highlights, colors, or otherwise visually indicates the zones that are “stressed” (e.g., zones with values on a stress scale above a threshold, zones with stress being one of the top three dispositions for the particular zone, etc.).

In still another example, a button may be included or a requester may otherwise select an input to obtain a zone disposition profile that is scaled in accordance with a personal profile of the requester. For instance, in one example, in addition to learning and storing zone disposition profiles, the present disclosure may also learn, store, and utilize personal profiles of requesters. To illustrate, a requester may have a unique perspective with different opinions from others as to what is considered “vibrant,” what is considered “stressed,” what is considered “abrasive,” what is considered “very vibrant” or “very stressed,” etc. The requester's perspective may be cultural, may be formed based upon a type of region in which the requester lived as a child, a type of region in which the requester has most recently lived, a marital status, a status of a number of children (or having no children), may be formed based upon general personality characteristics of the requester (e.g., introverted vs. extroverted, curious vs. not curious, relaxed vs. stressed, etc.), and so forth.

In one example, the present disclosure may learn a requester's perspective (or “personal profile”) based upon user feedback regarding different zones that the requester may visit. For instance, the requester may be asked to rank/score a zone with respect to different dispositions. The requester's selections may then be compared to a zone disposition profile determined in accordance with the present disclosure to learn how the requester's perspective may diverge from what is discovered via sensor data described above. For instance, if a zone disposition profile indicates that a zone is considered “very vibrant” and the requester has ranked the zone as “somewhat vibrant,” the present disclosure may determine that the requester's opinion as to what is “very vibrant” requires “more vibrancy” than average (or at least more than what is determined via sensor data as described above). As more feedback data is obtained from a requester visiting various zones, the requester's perspective may be learned with increased confidence. In addition, the requester's perspective may be applied to “scale” the results that may be presented as a zone disposition profile. For instance, in the example screen 230, the marker for vibrancy may be moved to the left/down the scale to indicate that while zone 2 is considered “very vibrant” in general, the requester may find it less so (and similarly for other dispositions in the disposition profile). Thus, these and other modification are all contemplated within the scope of the present disclosure.

FIG. 3 illustrates a flowchart of an example method 300 for reporting a disposition of a first zone identified based upon sensor data from a plurality of sensor devices applied to at least one detection model, in accordance with the present disclosure. In one example, the method 300 is performed by a component of the system 100 of FIG. 1, such as by server(s) 116, application server 104, and/or any one or more components thereof (e.g., a processor, or processors, performing operations stored in and loaded from a memory), by server(s) 116 and/or application server 104 in conjunction with one or more other devices, such as DB 106, DB(s) 118, or any of the sensor devices of FIG. 1, and so forth. In one example, the steps, functions, or operations of method 300 may be performed by a computing device or system 400, and/or processor 402 as described in connection with FIG. 4 below. For instance, the computing device or system 400 may represent any one or more components of a device, server, and/or application server in FIG. 1 that is/are configured to perform the steps, functions and/or operations of the method 300. Similarly, in one example, the steps, functions, or operations of method 300 may be performed by a processing system comprising one or more computing devices collectively configured to perform various steps, functions, and/or operations of the method 300. For instance, multiple instances of the computing device or processing system 400 may collectively function as a processing system. For illustrative purposes, the method 300 is described in greater detail below in connection with an example performed by a processing system. The method 300 begins in step 305 and may proceed to optional step 310, optional step 320, or step 330.

At optional step 310, the processing system may train at least one detection model (e.g., at least one machine learning model (MLM) or other detection model, such as a SURF or SIFT feature model, etc.) via a training data set, the training data set comprising at least one of: video samples or audio samples labeled with respect to at least one disposition from a defined set of dispositions. In one example, step 310 may include training various detection models for different semantic concepts, which may include dispositions, or other semantic concepts that can be mapped to dispositions.

At optional step 320, the processing system may associate values on at least one of: a water quality scale or an air quality scale with respective dispositions from a defined set of dispositions. For example, optional step 320 may comprise performing a regression analysis to learn a relation between water quality values or air quality values as predictors, and a respective disposition from the defined set of dispositions as an outcome. In one example, the regression analysis may be based on a training data set comprising at least one of: water quality measurements or air quality measurements and associated dispositions from the defined set of dispositions. In one example, different regression analyses may be performed for different dispositions. In the case where multiple sensor data are inputs, the regression analysis may be a multiple regression analysis (MRA). In one example, the result of regression is a prediction model (e.g., a MLM) for predicting one or more dispositions of a zone based upon new sensor data from the zone. In one example, the associated dispositions may be determined via one or more detection models based on at least one of camera or microphone input data (and which may be temporally associated with the water quality measurements, air quality measurements, etc.). Alternatively, or in addition, the associated dispositions may be determined via surveys, interviews, or the like. For example, individuals may be used as proxies for a disposition profile of a zone. Thus, these individuals may be used as training examples from which inferred zone profiles may be learned from the predictors.

At step 330, the processing system collects sensor data for a first zone via a plurality of sensor devices deployed in the first zone in communication with the processing system, where the plurality of sensor devices comprises at least one of: a camera or a microphone, and where the sensor data is collected over a period of time.

At step 340, the processing system identifies that a first disposition is associated with the first zone based upon the sensor data. For instance, step 340 may comprise applying at least one detection model to the sensor data, wherein the at least one detection model is configured to output at least one disposition based upon the sensor data as input data to the at least one detection model. For instance, the detection model may be trained/generated at optional step 310 above, or may be otherwise obtained by the processing system for use in connection with the method 300. The first disposition may comprise a representative temperament or personality of people within the zone (also referred to herein as mood, mental state, and/or emotional state). As noted above, the at least one disposition may be a disposition from a defined set of dispositions. In one example, the at least one detection model may be a detection model for the at least one disposition. In this regard, it should be noted that the at least one disposition may comprise/include the first disposition. In one example, the at least one detection model is to detect features of a human face in the sensor data (e.g., image data from a camera) and to output the at least one disposition based upon the features of the human face, e.g., human facial expression.

In one example, the at least one detection model may comprise a plurality of detection models, where each disposition of the defined set of dispositions has an associated detection model of the plurality of detection models. In one example, the processing system thus implements the plurality of detection models, and the identifying at step 340 may be in accordance with the plurality of detection models. In this regard, it should be noted that the sensor data collected over the period of time at step 330 may comprise a plurality of inputs to the at least one detection model, and step 340 may comprise aggregating a plurality of outputs of the at least one detection model.

For instance, the aggregating may comprise tallying the plurality of outputs associated with each of a plurality of dispositions from a defined set of dispositions. For example, the first disposition may comprise a disposition from the defined set of dispositions having a highest tally count (or from among the top three dispositions, the top four dispositions, etc.), a disposition having a score, tally, or the like above a threshold, and so forth. In other words, the first disposition may be identified as being associated with the zone (e.g., characteristic or representative of the zone) when the first disposition is has a higher tally count that for other dispositions. In one example, the first disposition may be identified as being associated with the first zone when a threshold number or percentage of the plurality of outputs comprises the first disposition. For example, the threshold number or percentage may be based upon a total number of the plurality of inputs. For instance, the processing system may establish that a minimum number of samples of sensor data/input data is required before any dispositions may be considered representative of a zone. In one example, the minimum number of samples may be fixed, or may be based upon a number of people estimated to be present in the zone. For instance, if the first zone is estimated to have 10,000 people, a minimum of 5,000 samples, 10,000 samples, etc. may be required (e.g., 0.5 samples per one person, 1 sample per 1 person, etc.).

In one example, the at least one detection model may comprise a plurality of detection models for detecting different semantic concepts in image or audio data, and for mapping different detected sematic concepts into one or multiple dispositions (e.g., detected sports and recreation can be mapped to vibrant, healthy, etc.). In one example, this mapping may be considered a last stage of a detection model, where the base detection model determines a semantic concept, the semantic concept is mapped to disposition(s), and the ultimate output is the at least one disposition. In one example relating to a plurality of detection models, a first portion of the sensor data from the camera or the microphone may be applied to a first detection model and a second portion of the sensor data from at least one additional sensor device may applied to a second detection model. For instance, the second detection model may be generated at optional step 320 as discussed above. In one example, step 340 may include combining outputs of the first detection model and the second detection model to generate an ensemble or collective output (e.g., where the ensemble output comprises the at least one disposition).

In one example, the combination may be a ratio-based combination, e.g., depending upon the amount of image or audio data that may be collected, depending upon the number of disposition related events found in the image or audio data (e.g., there may be continuous video or audio, but not many people present, which can indicate an abundance of personal space, but does little to tell whether the people are speaking or acting happy, angry, stressed, etc.), and so on.

At step 350, the processing system reports that the first disposition is associated with the first zone. For instance, in one example, a report may be generated or provided in response to a request from a user device, such as a device of city planner, a potential visitor to the area, a potential home purchaser, and so forth. In one example, step 350 may comprise generating a map of an area including the first zone, where the first disposition being associated with the first zone may be indicated in relation to the zone on the map (e.g., via shading, color coding with a dot, border, or other markers, a dialog box pointing toward or otherwise clearly showing association with the first zone, etc.). In one example, the first disposition may be presented with one or more other dispositions in a disposition profile of the first zone. In one example, the map may show other zones in the area, e.g., a second zone, a third zone, etc. and their respective dispositions (e.g., disposition profiles indicating respective disposition(s) associated with each zone). Step 350 may alternatively or additionally include reporting (or storing) the first disposition in another manner such as illustrated in FIG. 2 or the like.

Following step 350, the method 300 proceeds to step 395 where the method ends.

It should be noted that the method 300 may be expanded to include additional steps, or may be modified to replace steps with different steps, to combine steps, to omit steps, to perform steps in a different order, and so forth. For instance, in one example, the processing system may repeat steps 330 and 340 for various instance of sensor data from the same or different sensors in the first zone. In addition, step 340 may comprise or the method 300 may include an additional step of aggregating various dispositions determined from multiple instances of sensor data to generate a disposition profile of the first zone. In one example, the processing system may repeat one or more steps of the method 300 for a different area or zone. In one example, the method 300 may include registering sensors into the sensor database. In one example, the method 300 may include quantifying the dispositions of one or more test zones (e.g., disposition profiles) via surveys, interviews, etc., and then training/generating machine learning models or other detection models on video and/or audio data to predict a disposition of a zone with a target accuracy (e.g., 65 percent accurate, 80 percent accurate, 90 percent accurate, etc.) after the collection of X number of samples. In such an example, step 340 may include adjusting a weighting ratio between dispositions determined from one type of sensor data versus another. For instance, when analyzing the first zone and there are less than X number of video or audio samples and/or less than X number of detected events, the ratio may be adjusted to rely more upon the predictions from additional sensor device(s). This may be on a sliding scale based upon the number of video or audio samples and/or the number of detected events from such video or audio samples.

In one example, the method 300 can include defining zones based upon landmarks, or accepting one or more inputs for user-defined neighborhoods or zones. For instance, city planners may use this results from step 350 for various purposes and may define a neighborhood as a zone for investigative purposes. In another instance, the results can be used to ascertain the mood of a large gathering of people to detect potential security or safety risks, e.g., the mood of a large crowd celebrating a sporting event outcome. However, in another example, this can automatically be done by the processing system to account for population density or perceived population density, a perceived basis for geographic grouping (or multiple factors indicative of geographic grouping), a number of available sensors in an area, etc. In one example, the method 300 may include obtaining and providing at step 350 additional data along with disposition information, such as a type of area (residential, commercial, office, industrial, recreational, etc.), an estimated density of people in the zone, e.g., based upon existing available census and demographic data or estimated in other ways, such as average number of unique detected mobile endpoint devices in the zone, and so forth. In one example, density of people may be broken down by morning, afternoon, evening, or hours of the day, days of the week, days, months, seasons or other times of the year, and so forth. In such an example, the method 300 may include obtaining a user/requester selection of a time period of interest for the reporting.

In one example, the method 300 may include detecting noise (e.g., average noise level over a day) from microphone(s), and/or detecting specific noise stressors, e.g., highway traffic, airplane noise, helicopter noise, train noise, landscaping noise, construction noise, etc., which may impact the disposition. These may be types of semantic content that can be mapped to disposition scales, rather than a separate category of inputs, or can be additional inputs to ensemble detection models. In one example, additional sensor data may include data from infrastructure vibration sensors (e.g., where infrastructure can include bridges, buildings, etc.), which may be associated to one or more dispositions at optional step 320.

In one example, additional sensor data such as precipitation, temperature, and humidity may be associated with dispositions and may affect the disposition profile of a zone that may be determined in accordance with the method 300. It should be noted that there may be long standing stereotypes about weather and mood. However, it is important to note that these may be primarily speculative and far from universally applicable. In addition, the dispositions of people from zone to zone, even nearby, may change dramatically. For instance, those in a closed valley may be less content versus a neighborhood on the crest of the hill with ocean views, even if all are subject to more rainfall throughout the year that those living in another region. Thus, while precipitation, temperature, and humidity may have some effect on disposition, these are merely additional factors that may be in addition to air quality or water quality, as well as the primary factors of camera and/or microphone data. In one example, the method 300 may be expanded or modified to include steps, functions, and/or operations, or other features described above in connection with the example(s) of FIGS. 1 and 2, or as described elsewhere herein. Thus, these and other modifications are all contemplated within the scope of the present disclosure.

In addition, although not expressly specified above, one or more steps of the method 300 may include a storing, displaying and/or outputting step as required for a particular application. In other words, any data, records, fields, and/or intermediate results discussed in the method can be stored, displayed and/or outputted to another device as required for a particular application. Furthermore, operations, steps, or blocks in FIG. 3 that recite a determining operation or involve a decision do not necessarily require that both branches of the determining operation be practiced. In other words, one of the branches of the determining operation can be deemed as an optional step. Furthermore, operations, steps or blocks of the above described method(s) can be combined, separated, and/or performed in a different order from that described above, without departing from the example embodiments of the present disclosure.

FIG. 4 depicts a high-level block diagram of a computing device or processing system specifically programmed to perform the functions described herein. For example, any one or more components or devices illustrated in FIG. 1 or described in connection with the examples of FIG. 2 or 3 may be implemented as the processing system 400. As depicted in FIG. 4, the processing system 400 comprises one or more hardware processor elements 402 (e.g., a microprocessor, a central processing unit (CPU) and the like), a memory 404, (e.g., random access memory (RAM), read only memory (ROM), a disk drive, an optical drive, a magnetic drive, and/or a Universal Serial Bus (USB) drive), a module 405 for reporting a disposition of a first zone identified based upon sensor data from a plurality of sensor devices applied to at least one detection model, and various input/output devices 406, e.g., a camera, a video camera, storage devices, including but not limited to, a tape drive, a floppy drive, a hard disk drive or a compact disk drive, a receiver, a transmitter, a speaker, a display, a speech synthesizer, an output port, and a user input device (such as a keyboard, a keypad, a mouse, and the like).

Although only one processor element is shown, it should be noted that the computing device may employ a plurality of processor elements. Furthermore, although only one computing device is shown in the Figure, if the method(s) as discussed above is implemented in a distributed or parallel manner for a particular illustrative example, i.e., the steps of the above method(s) or the entire method(s) are implemented across multiple or parallel computing devices, e.g., a processing system, then the computing device of this Figure is intended to represent each of those multiple general-purpose computers. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented. The hardware processor 402 can also be configured or programmed to cause other devices to perform one or more operations as discussed above. In other words, the hardware processor 402 may serve the function of a central controller directing other devices to perform the one or more operations as discussed above.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computing device, or any other hardware equivalents, e.g., computer readable instructions pertaining to the method(s) discussed above can be used to configure a hardware processor to perform the steps, functions and/or operations of the above disclosed method(s). In one example, instructions and data for the present module or process 405 for reporting a disposition of a first zone identified based upon sensor data from a plurality of sensor devices applied to at least one detection model (e.g., a software program comprising computer-executable instructions) can be loaded into memory 404 and executed by hardware processor element 402 to implement the steps, functions or operations as discussed above in connection with the example method(s). Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 405 for reporting a disposition of a first zone identified based upon sensor data from a plurality of sensor devices applied to at least one detection model (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not limitation. Thus, the breadth and scope of a preferred embodiment should not be limited by any of the above-described example embodiments, but should be defined only in accordance with the following claims and their equivalents.

What is claimed is:

1. A method comprising:
collecting, by a processing system including at least one processor, sensor data for a first zone via a plurality of sensor devices deployed in the first zone in communication with the processing system, wherein the plurality of sensor devices comprises at least one of: a camera or a microphone, wherein the sensor data is collected over a period of time;
identifying, by the processing system, that a first disposition is associated with the first zone based upon the sensor data, wherein the identifying comprises applying at least one detection model to the sensor data, wherein the at least one detection model is configured to output at least one disposition based upon the sensor data as input data to the at least one detection model, wherein the at least one disposition comprises the first disposition, wherein the sensor data collected over the period of time comprises a plurality of inputs to the at least one detection model, and wherein the identifying that the first disposition is associated with the first zone comprises aggregating a plurality of outputs of the at least one detection model from the plurality of inputs; and
reporting, by the processing system, that the first disposition is associated with the first zone.

2. The method of claim 1, wherein the first disposition comprises a representative a temperament of people within the first zone.

3. The method of claim 1, wherein the at least one disposition is identified from a defined set of dispositions.

4. The method of claim 3, wherein the at least one detection model comprises a plurality of detection models.

5. The method of claim 4, wherein each disposition of the defined set of dispositions has an associated detection model of the plurality of detection models.

6. The method of claim 5, wherein the processing system implements the plurality of detection models, and wherein the identifying is in accordance with the plurality of detection models.

7. The method of claim 1, wherein the plurality of sensor devices comprises the camera, and wherein the at least one detection model is to detect features of a human face in the sensor data, the sensor data comprising image data from the camera, and to output the at least one disposition based upon the features of the human face.

8. The method of claim 1, wherein the first disposition is identified as being associated with the first zone when a threshold number or percentage of the plurality of outputs comprises the first disposition.

9. The method of claim 8, wherein the threshold number or percentage is based upon at least one of:
a total number of the plurality of inputs; or
a total number of the plurality of inputs in relation to a number of people estimated to be in the first zone.

10. The method of claim 1, wherein the aggregating comprises tallying the plurality of outputs associated with each of a plurality of dispositions from a defined set of dispositions, wherein the first disposition comprises a disposition from the defined set of dispositions having a highest tally count.

11. The method of claim 1, further comprising:
training the at least one detection model via a training data set, the training data set comprising at least one of: video samples or audio samples labeled with respect to the at least one disposition.

12. The method of claim 1, wherein the plurality of sensor devices further comprises at least one additional sensor device comprising at least one of:
a water quality sensor; or
an air quality sensor.

13. The method of claim 12, further comprising:
associating values on at least one of: a water quality scale, or an air quality scale, with respective dispositions from a defined set of dispositions.

14. The method of claim 13, wherein the associating comprises performing a regression analysis to learn a relation between at least one of: water quality values or air quality values as predictors and a respective disposition from the defined set of dispositions as an outcome.

15. The method of claim 14, wherein the regression analysis is based on a training data set comprising at least one of: water quality measurements or air quality measurements and associated dispositions from the defined set of dispositions.

16. The method of claim 12, wherein the at least one detection model comprises a plurality of detection models, wherein a first portion of the sensor data from the at least one of: the camera or the microphone is applied to a first detection model of the plurality of detection models and wherein a second portion of the sensor data from the at least one additional sensor device is applied to a second detection model of the plurality of detection models.

17. The method of claim 16, wherein outputs of the first detection model and the second detection model are combined to generate an ensemble output, wherein the ensemble output comprises the at least one disposition.

18. The method of claim 1, wherein the reporting comprises generating a map of an area including the first zone, and wherein the first disposition of the first zone is indicated in relation to the first zone on the map.

19. A non-transitory computer-readable medium storing instructions which, when executed by a processing system including at least one processor, cause the processing system to perform operations, the operations comprising:
collecting sensor data for a first zone via a plurality of sensor devices deployed in the first zone in communication with the processing system, wherein the plurality of sensor devices comprises at least one of a camera or a microphone, and wherein the sensor data is collected over a period of time;
identifying that a first disposition is associated with the first zone based upon the sensor data, wherein the identifying comprises applying at least one detection model to the sensor data, wherein the at least one detection model is configured to output at least one disposition based upon the sensor data as input data to the at least one detection model, wherein the at least one disposition comprises the first disposition, wherein the sensor data collected over the period of time comprises a plurality of inputs to the at least one detection model, and wherein the identifying that the first disposition is associated with the first zone comprises aggregating a plurality of outputs of the at least one detection model from the plurality of inputs; and
reporting that the first disposition is associated with the first zone.

20. An apparatus comprising:
a processing system including at least one processor; and
a computer-readable medium storing instructions which, when executed by the processing system, cause the processing system to perform operations, the operations comprising:
collecting sensor data for a first zone via a plurality of sensor devices deployed in the first zone in communication with the processing system, wherein the plurality of sensor devices comprises at least one of a camera or a microphone, and wherein the sensor data is collected over a period of time;
identifying that a first disposition is associated with the first zone based upon the sensor data, wherein the identifying comprises applying at least one detection model to the sensor data, wherein the at least one detection model is configured to output at least one disposition based upon the sensor data as input data to the at least one detection model, wherein the at least one disposition comprises the first disposition, wherein the sensor data collected over the period of time comprises a plurality of inputs to the at least one detection model, and wherein the identifying that the first disposition is associated with the first zone comprises aggregating a plurality of outputs of the at least one detection model from the plurality of inputs; and
reporting that the first disposition is associated with the first zone.
